(19) United States
(12) Patent Application Publication
(10) Pub. No.: US 2002/0177171 A1
(43) Pub. Date: Nov. 28, 2002

(54) Method and means for caries prevention and susceptibility detection

(75) Inventor: Nicklas Stromberg, Umea (SE)

Correspondence Address: DICKSTEIN SHAPIRO MORIN & OSHINSKY, LLP, 1177 Avenue of the Americas, New York, NY, 10036, US

(73) Assignee: Conimar AB

(21) Appl. No.: 10/206,193

(22) Filed: Jul. 29, 2002

Related U.S. Application Data
(62) Division of application No. 09/701,407, filed on Dec. 22, 2000.

(30) Foreign Application Priority Data
May 28, 1998 (SE) 9801893-0

(51) Int. Cl. G01N 33/53

(52) U.S. Cl. 435/7.1

(57) ABSTRACT

A method for identifying the susceptibility of a person to dental caries comprises the assay of one or several of: an agent having high binding capacity to Streptococcus mutans but low binding capacity to Actinomyces naeslundii, an agent having high binding capacity to S. mutans-binding agglutinin, an agent having high binding capacity to A. naeslundii. The agent having high binding capacity to Streptococcus mutans but low binding capacity to A. naeslundii comprises an allelic Db type of salivary acidic proline-rich molecules (acidic PRPs), a biologically active fragment or a genetic precursor thereof. A corresponding reagent is also identified. Also disclosed are a method and an agent for the prevention of caries, as well as labelled S. mutans and A. naeslundii and their use.

This is a division of application Ser. No. 09/701,407, filed Dec. 22, 2000.

### FIELD OF THE INVENTION

The present invention relates to a method for determining the susceptibility of individuals to dental caries and a corresponding means. The present invention also relates to a method for preventing caries and a corresponding means.

### BACKGROUND OF THE INVENTION

The two major dental diseases, dental caries and periodontitis, are chronic polymicrobial infectious diseases [1,2,3]. Two principal mechanisms, adhesion and metabolism, account for the oral polymicrobial societies. Acid proline-rich proteins (acidic PRPs: PRP-1; PRP-2; Db; PIF; Pa) from the PRH1 and PRH2 gene loci on chromosome 12p13.2, act at the tooth-saliva interface (4,5]. Acidic PRPs regulate calcium phosphate precipitation, crystal formation and attach commensal Actinomyces and streptococci over pathogenic S. mutans and lactobacilli. The acidic PRPs are polymorphic proteins displaying both allelic (large PRP-1, PRP-2 PIF-s, Db-s and Pa-s) and post-translatory (small PRP-3, PRP-4, PIF-f and DB-f) variation.

In spite of the general decline in caries in most Western countries during recent decades 5-10 per cent of the population is still highly diseased. While life-style is a determinant factor in the development of caries a substantial number of individuals with good oral hygiene nevertheless develop caries while other individuals with mediocre or no oral hygiene to speak of seem to be quite resistant to the disease. mediocre or no oral hygiene to speak of seem to be quite resistant to the disease. The cost to society of remedying the effects of caries is huge. Early treatment would substantially reduce this cost. The problem is, however, that a person having caries usually does not become aware of it until a late stage. While frequent controls of the entire population by dentists would provide an obvious solution to the problem such controls per se would entail considerable cost. A better way would be to identify the persons at risk, in particular persons at risk in spite of good oral hygiene.

### OBJECTS OF THE INVENTION

It is an object of the present invention to provide a method for identifying persons at risk for developing canes not related to oral hygiene. It is another object of the present invention to provide a means for use in this method.

It is a further object of the present invention to provide a method for preventing caries. It is another object of the present invention to provide a corresponding means.

Further objects of the invention will become apparent from the following short description of the invention and a preferred embodiment of it, as well as of the appended claims.

### SHORT DESCRIPTION OF THE INVENTION

According to the present invention is disclosed a method for identifying the susceptibility of a person to dental caries by assaying, in a saliva sample, one or several of: an agent having high binding capacity to Streptococcus mutans but low binding capacity to Actinomycetes naeslundii, an agent having high binding capacity to S. mutans-binding agglutinin, an agent having high binding capacity to A. naeslundii.

The invention is based on the finding that multivariate PLS-modelling showed the allelic Db type, but not other acidic PRPs, to coincide with high saliva adhesion capacity of cariogenic Streptococcus mutans and high caries experience. In contrast, the total amount and most types of acidic PRP, but not Db, coincided with high saliva adhesion capacity of A. naeslundii and low caries experience. On the other hand, a high adhesion capacity of A. naeslundii is indicative for low caries experience, and thus a negative marker in respect of the risk for a person of developing caries. Moreover it is considerably simpler to type for PRP-adhesion than adhesion of S. mutans since the former method can be reliably carried out even after a thousand-fold dilution of the saliva which is not the case with the latter.

The agent having high binding capacity to S. mutans but low binding capacity to A. naeslundii preferably comprises an allelic Db type of salivary acidic proline-rich molecules (acidic PRPs), a biologically active fragment or a genetic precursor thereof.

The agent having high binding capacity to A. naeslundii preferably comprises PRP2.

It is preferred for the method of the invention to comprise the procurement of a saliva sample which is assayed for S. mutans-binding or A. naeslundii-binding by means of a monoclonal antibody.

It is also preferred for the method of the invention to comprise the procurement of a saliva sample which is assayed for S. mutans-binding or A. naeslundii-binding by a PCR technique.

According to an aspect of the invention the method comprises the determination of the saliva mediated adherence of S. mutans or A. naeslundii to a support coated with saliva procured from said person. In particular a hydroxyapatite support is preferred.

Also disclosed is reagent for the determination of the susceptibility of a person to dental caries comprising an agent selected from agent having high binding capacity to S. mutans but low binding capacity to A. naeslundii, agent having high binding capacity to S. mutans-binding agglutinin, agent having high binding capacity to A. naeslundii. The reagent preferably comprises an allelic Db type of salivary acidic proline-rich molecules (acidic PRPs), a biologically active fragment or a genetic precursor thereof.

According to another aspect of the invention is disclosed a method for the prevention of caries in a person, comprising administration to said person of an agent promoting the competitive adherence of A. naeslundii over S. mutans to dental surfaces.

According to a further aspect of the invention is disclosed an agent for the prevention of caries in a person comprising an antibody having substantial binding capacity in regard to S. mutans but not to A. naeslundii. Preferably the specific binding capacity of the agent in regard to S. mutans exceeds its specific binding capacity in respect of A. naeslundii by a factor of ten or more.

As a reagent for assessing the risk for a person developing caries the invention in particular teaches the use of labelled A. naeslundii or S. mutans, preferably labelled with a scintillation label, having substantial capacity to bind to the allelic Db-type of acidic PRPs or PRP-2, respectively.

### BRIEF DESCRIPTION OF THE DRAWING

Also disclosed is the use of labelled A. naeslundii or S. mutans for identifying the susceptibility of a person to dental caries.

### DESCRIPTION OF THE DRAWING

In the following the invention will be explained in greater detail by reference to a preferred embodiment illustrated by a drawing comprising a single FIGURE which showing the distribution of dental caries experience in 3,400 Swedish 12 year old subjects.

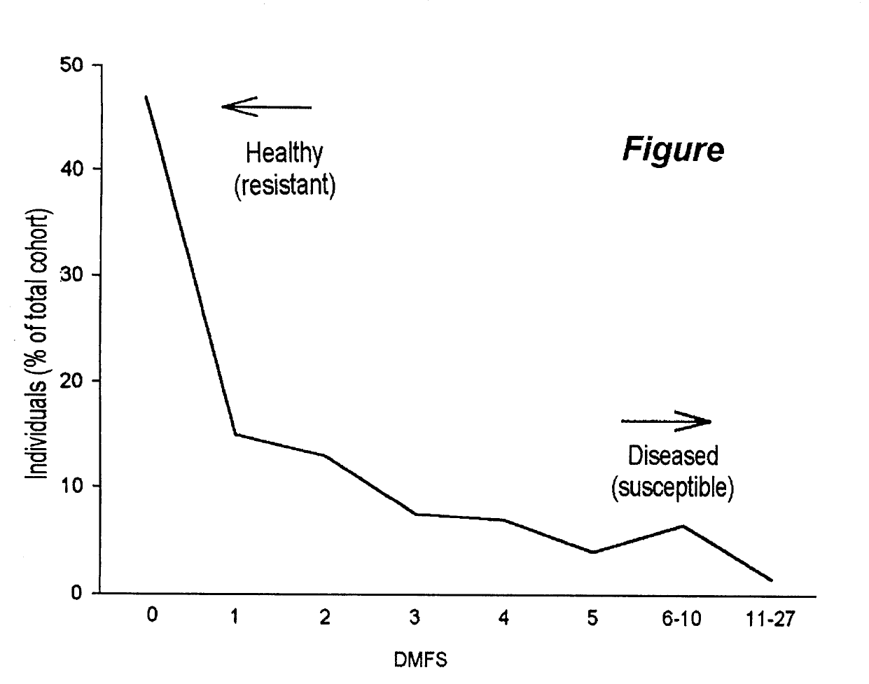

In a first step to link acidic PRP polymorphism to dental caries, 19 children with high (mean DMFS=5,0) and 19 subjects with low (mean DMFS=0) caries experience were randomly selected from a cohort of 500 subjects from Northern Sweden (FIG. 1). DMFS=total number of Decayed, Missing and Filled Tooth Surfaces. The cohort is part of an ongoing nation-wide epidemiological survey comprising 3,400 12-year-old children showing a markedly skewed caries experience but odds ratios of only 2-3 for traditional life-style factors such as sugar intake, fluoride exposure and oral hygiene. Similarly, markers for dietary habits (sugar intake, sweet snacks and soft drinks), fluoride exposure and oral hygiene displayed odds ratios of 2-3 for the low versus high caries subjects (Table 1).

The rather uniform distribution of life-style factors in the whole cohort and the group of extremes supports a role of genetic polymorphism in host susceptibility and resistance to dental caries in explaining different caries experiences.

By electrophoretic analysis of saliva and multivariate PLS-modelling the amount and allelic types of acidic PRPs were related to caries experience at base-line and after a 2-year follow-up (Table 1). The allelic Db variant coincided significantly with a high caries experience (VIP 1.25; odds ratio 3, Table 1). In contrast, the other types of acidic PRPs (Pa, PRP-1/PIF-s and PRP-2) coincided, although not significantly, with a low caries experience. In addition, the amount of acidic PRPs, irrespective of allelic types, coincided significantly with a low caries experience (Table 1).

For investigating a mechanistic model explaining the role of acidic PRP polymorphism in caries susceptibility, a relationship was established between amount and types of acidic PRPs and the saliva adhesion capacity of commensal (A. naeslundii) and cariogenic (S. mutans) bacteria (Table 2). Most acidic PRP types, but not Db, coincided significantly with the saliva adhesion capacity of A. naeslundii strain LY7, which binds to acidic PRPs. In contrast, the Db type coincided significantly, but less strongly than S. mutans-binding glycoprotein, with the saliva adhesion capacity of S. mutans strain Ing-Britt (Table 2). Furthermore, the saliva adhesion capacity of A. naeslundii strain LY7 and S. mutans strain Ing-Britt coincided significantly with a low and high caries experience, respectively. In contrast, the saliva adhesion capacity of A. naeslundii strain P-4-N, binding to β-linked galactosamine structures, did not coincide with caries experience. Thus, there is a link between acidic PRPs and the saliva adhesion capacity of commensal and cariogenic bacteria, a property linked to dental caries experience.

The present findings showing a link between acidic PRP polymorphism, bacterial adhesion and caries experience demonstrate a role of genetic polymorphism in susceptibility and resistance toward dental caries. The odds ratios of typical life style factors were only 2-3 for the high versus low caries groups, holding true for the whole cohort and the present groups of extremes, and of similar magnitudes to those observed for acidic PRP polymorphism or bacterial adhesion. Since the saliva adhesion capacity of S. mutans coincided most strongly with caries experience, and since S. mutans-binding glycoprotein explained this adhesion better than Db, susceptibility to dental caries is likely to involve multiple salivary factors. Nonetheless, the present findings point to the possibility of allelic and post-translatory modifications of host adhesion molecules modulating the microbial ecology at tooth surfaces. It is noteworthy that acidic PRPs are multifunctional proteins, which could modulate the ecology by several mechanisms. Furthermore, the Db variant, encoded by the PRH2 locus, is structurally different to the other allelic variants by virtue of a central amino acid repeat. Db seems to have a unique function. For example, the Db protein may hold a configuration or conformation masking the carboxy-terminal ProGln motif targeted by A. naeslundii, explaining why some acidic PRP types, but not Db, coincide with the saliva adhesion capacity of this micro-organism. It may, at the same time, expose a site interacting with S. mutans-binding agglutinin, explaining why it coincides with the saliva adhesion capacity of S. mutans. Similarly, conformational modifications of host carbohydrates has been proposed to select for different bacterial types, and heterophilic interactions are well-known among salivary and mucus components.

Material and Methods

Study group. Thirty-eight 12-year-olds (20 boys, 18 girls) were randomly selected from a cohort of 500 subjects in the northern part of Sweden, being part of a nation-wide epidemiological survey of 3,400 children. Stimulated parotid saliva and whole saliva were sampled from each subject, and analysed for numbers of S. mutans and lactobacilli, all according to standardised routines. The DMFs index Within the framework of the basic study, data on socio-economic status, dietary habits, oral hygiene and use of fluoride were gathered by a questionnaire. Acidic proline-rich proteins were analyzed by native electrophoresis in discontinuous 4.2-7.1% polyacrylamide as described earlier [6]. The allelic PIF variants were assessed by isoelectric focusing [7]. The gels were scanned on a Model GS-700 Imaging Densitometer (Bio-Rad, Hercules, Calif.) and the Molecular Analyst® software (Bio-Rad) was used for densitometric analyses. S. mutans-binding agglutinin was analysed by gel electrophoresis (see [9] below and quantified in a slot-blot assay using monoclonal antibody mAb143 (see also [9]).

Bacterial adhesion. Saliva-mediated adherence of Actinomyces naeslundii strains LY7 and P-4-N and S. mutans strain Ing-Britt was measured as described earlier [8]. Briefly, 5 mg of hydroxyapatite beads (Fluka, Chemika, Fluka Chemie AG, Buchs, Switzerland, 0.08-0.20 mm) coated with saliva, were incubated with 125 μl of 35S-methionine labelled bacteria (105 cpm/ml and 5×108 bacteria/ml). The number of bacteria remaining attached to the beads after repeated washes was estimated by scintillation counting of the beads.

While the acidic proline-rich protein (acidic PRPs) variants were measured by electrophoretic techniques their reliability was confirmed by HPLC and FPLC [3,9]. The total amounts of acidic PRPs were both analysed by electrophoretic means and by FPLC.

The electrophoretic techniques, designed to specifically resolve the acidic proline-rich proteins (PRPs), were performed as described in the literature [6,7]. Upon standard treatment, the parotid saliva sample was subjected to native alkaline electrophoresis in discontinuous 4-7% polyacrylamide gels which detect most allelic and post-translatory variants of acidic PRPs. Isoelectric focusing (pH 3.5-6.0) was performed to specifically detects the PIF variant. The resolved protein bands were chemically stained according to standard techniques, involving 0.05% Coomassie Brilliant Blue in 20% trichloroacetic acid (TCA) for 16 h and destained slowly in 2% acetic acid. The stained PRP protein variants were identified by means of mobility and scanned into a computer for densitometric determinations of the amount of protein. The amounts of PRP variants were determined against a standard curve of varying PRP concentrations and amounts.

The electrophoretic method was shown to give essentially the same results as HPLC analysis of parotid saliva samples of defied individuals. The HPLC method, specifically designed to resolve PRP variants, were performed as described in the literature [3]. After standard treatment, about 20 μl of saliva was injected into an GenPak FAX HPLC anion-exchange column using a commercial HPLC system. The proteins were eluted using a salt gradient and identified by means of retention times.

The amount of total PRPs were also determined by FPLC protein separation of the salivae on anion exchange —N⁺(CH)₃ column (Bio-Scale Q2, Bio-Rad, Hercules, Calif.) using the BioLogic system (Biologic™, Bio-Rad) for controlling and monitoring the chromatography. Saliva samples were diluted once in buffer A (25 mmol/L NaCl in 50 mmol/L Tris-HCl, pH 8.0) centrifuged at 9,000×g for 10 min at 4° C. and aliquots (200 μL) were injected. The column was then washed with 4 column volumes of buffer A, and the proteins were eluted with a linear salt gradient from 25 mmol/L to 1.35 mol/L NaCl in the Tris-HCI buffer at a flow rate of 0.8 mL/min. Fractions of 0.4 mL were collected and the absorbance of the eluate was continuously monitored at 214 nm. The peak fit program was used to define and calculate the peak areas.

Alternative ways to determine the amount and type of PRP variants include methods using monoclonal antibodies or antisera to specific acid PRP variants in ELISA assays. Alternatively, PCR and specific primer pairs could be used to type the individual pattern of acidic PRPs.

Data management and statistical analysis. Multivariate statistics using the SIMCA® software package (version 6.0, Umetri AB, Umeå, Sweden) were applied. Partial Least Square Projections to latent structures were used. Before being entered into the multivariate models, skewed variables were logarithmically transformed and pre-processed by means of scaling and mean-centering. By this procedure, each marker was scaled to unit variance in order to be equally important in the analysis. Values of importance in the projection (VIP) were calculated. VIP-values are tools for variable selection and markers with VIP-values larger than 1.0 represent significant associations.

References

1. Strömberg, N. et al. Anti-adhesion and diagnostic strategies for oro-intestinal bacterial pathogens. In Toward Anti-Adhesion Therapy for Microbial Diseases. (eds. Kahane, I. & Ofek, I.) 9-24 (Plenum Press, New York, 1996).

2. Gibbons, R. J. Bacterial adhesion to oral tissues: a model for infectious diseases. J. Dent. Res. 68, 750-760 (1989).

3. Gustavsson, B. E. et al. The Vipeholm dental caries study. The effect of different levels of carbohydrate intake on caries activity in 436 individuals observed for five years. Acta Odont. Scand. 11, 232-364 (1954).

4. Hay, D. I., Ahern, J. M., Schluckebier, S. K. & Schlesinger, D. H. Human salivary acidic proline-rich protein polymorphisms and biosynthesis studied by high-performance liquid chromatography. J. Dent. Res. 73, 1717-1726 (1994).

5. Azen, E. A. Genetics of salivary protein polymorphisms. Crit. Rev. Oral Biol. Med. 4, 479-485 (1993).

6. Azen, E. A. & Yu, P.-L. Genetic polymorphism of PIF (Parotid isoelectric focusing variant) proteins with linkage to the PPP (Parotid proline-rich protein) gene complex. Biochemical Genetics. vol 19. No. 5/6. 1981.

7. Azen, E. A. & Denniston, C. Genetic polymorphism of PIF (parotid isoelectric focusing variant) proteins with linkage to the PPP (parotid proline-rich protein) gene complex. Biochem. Genet. 19, 475-485 (1981).

8. Gibbons, R. J. & Hay, D. I. Human salivary acidic proline-rich proteins and statherin promote the attachment of Actinomyces viscosus LY7 to apatitic surfaces. 56, 439-445 (1988).

9. Carlén, A. P., Bratt P., Stenudd C., Olsson J. and Strömberg, N. Agglutinin and acidic proline-rich protein receptor patterns may modulate bacterial adherence and colonization on tooth surfaces. J. Dent. Res. 77 (1) 1998.

What is claimed is

1. A method for identifying the susceptibility of a person to dental caries by assaying, in a saliva sample, one or several of:
an agent having high binding capacity to Streptococcus mutans but low binding capacity to Actinomyces naeslundii, an agent having high binding capacity to S. mutans-binding agglutinin, an agent having high binding capacity to A. Naeslundii.

2. The method of claim 1, wherein the agent having high binding capacity to S. mutans but low binding capacity to A. naeslundii comprises an allelic Db type of salivary acidic proline-rich molecules (acidic PRPs), a biologically active fragment or a genetic precursor thereof.

3. The method of claim 1, wherein the agent having high binding capacity to A. naeslundii comprises PRP-2.

4. The method of claim 1, comprising the procurement of a saliva sample which is assayed for S. mutans-binding or A. naeslundii-binding by means of a monoclonal antibody.

5. The method of claim 1, comprising the procurement of a saliva sample which is assayed for S. mutans-binding or A. naeslundii-binding by a PCR technique.

6. The method of claim 1, wherein the method comprises the determination of the saliva mediated adherence of S. mutans or A. naeslundii to a support coated with saliva procured from said person.

7. The method of claim 6, wherein the support comprises hydroxyapatite.

8. The method of claim 1, wherein the agent is labelled A. naeslundii or S. mutans.

9. A reagent for the determination of the susceptibility of a person to dental caries comprising an agent selected from agent having high binding capacity to S. mutans but low binding capacity to A. naeslundii, agent having high binding capacity to S. mutans-binding agglutinin, agent having high binding capacity to A. naeslundii.

10. The reagent of claim 9 comprising an allelic Db type of salivary acidic proline-rich molecules (acidic PRPs), a biologically active fragment or a genetic precursor thereof.

11. A method for the prevention of caries in a person, comprising administration to said person of an agent promoting the competitive adherence of A. naeslundii over S. mutans to dental surfaces.

12. Labelled A. naeslundii or S. mutans, in particular labelled with a scintillation label, having substantial capacity to bind to the allelic Db-type of acidic PRPs.
